(19) United States
(12) Patent Application Publication
(10) Pub. No.: US 2026/0199411 A1
(43) Pub. Date: Jul. 16, 2026

(54) PROBIOTICS FOR TREATING AND/OR PREVENTING CONDITIONS ASSOCIATED WITH HELICOBACTER PYLORY COLONIZATION

(71) Applicant: INTERNATIONAL N&H DENMARK APS, Kongens Lyngby (DK)

(72) Inventors: DAFANG GAO, SHANGHAI (CN); JING YANG, SHANGHAI (CN); ARTHUR OUWEHAND, KANTVIK (FI); YANG MI, Zhengzhou (CN); SIQI SHEN, Zhengzhou (CN); PENGYUAN ZHENG, ZHENGZHOU (CN)

(21) Appl. No.: 19/133,366

(22) PCT Filed: Dec. 5, 2023
(86) PCT No.: PCT/EP2023/084238

§ 371 Date: May 28, 2025

(30) Foreign Application Priority Data
Dec. 5, 2022 (WO) PCT/CN2022/136639

(51) Int. Cl. A61K 35/747; A61K 31/05; A61K 31/4164; A61K 31/43; A61K 31/555; A61K 31/65; A61K 31/7048; A61K 31/716; A61P 1/00; A61P 29/00; A61P 31/04

(52) U.S. Cl. CPC A61K 35/747; A61K 31/05; A61K 31/4164; A61K 31/43; A61K 31/555; A61K 31/65; A61K 31/7048; A61K 31/716; A61P 1/00; A61P 29/00; A61P 31/04

(57) ABSTRACT

This invention relates to bacterial strains of the species Lactobacillus acidophilus and Lactiplantibacillus plantarum for use in treating and or preventing conditions associated with colonization by Helicobacter pylori (H. pylori) of the gastric mucosa, for use in inhibiting Helicobacter pylori (H. pylori), for use in treating and or preventing Helicobacter pylori (H. pylori) infection, for use in maintaining gastric microbiota homeostasis and for use in treating and preventing gastric inflammation in a subject. This invention further relates to compositions, food products, dietary supplements, or pharmaceutically acceptable formulations comprising said bacterial strains.

### INCORPORATION BY REFERENCE OF THE SEQUENCE LISTING

The present application is being filed along with a Sequence Listing in electronic format. The Sequence Listing is provided as a file entitled NB42096-WO-PCT_2.xml, created Dec. 4, 2023, which is 2,000 bytes in size. The information in the electronic format of the Sequence Listing is incorporated by reference in its entirety.

### FIELD OF THE INVENTION

This invention relates to bacterial strains of the species Lactobacillus acidophilus and Lactiplantibacillus plantarum for use in treating and or preventing conditions associated with colonization by Helicobacter pylori (H. pylori) of the gastric mucosa, in a subject. This invention further relates to compositions, food products, dietary supplements, or pharmaceutically acceptable formulations comprising bacterial strain of the species Lactobacillus acidophilus and Lactobacillus plantarum for use in treating and or preventing conditions associated with colonization by Helicobacter pylori (H. pylori) of the gastric mucosa, in a subject.

### BACKGROUND

In recent years, the infection rate of Helicobacter pylori (H. pylori) has been around 50%. Infection with Helicobacter pylori (H. pylori) may lead to chronic gastritis, peptic ulcer, gastric lymphoma originating from mucosa-associated lymphoid tissue (gastric MALT lymphoma) and even gastric cancer. Helicobacter pylori has been widely defined as the critical carcinogenesis of gastric tissue. At present, the first line therapy in eradicating Helicobacter pylori (H. pylori) is the quadruple therapy combining a proton pump inhibitor, 2 antibiotics along with a bismuth agent.

However, due to the over administration and abuse of antibiotics, the resistance rate of Helicobacter pylori (H. pylori) to antibiotics has been increasing dramatically. Furthermore, potential side effects associated with some of the drugs used in the treatment of Helicobacter pylori (H. pylori), such as headaches, nausea, vomiting, gastrointestinal flora dysbiosis and even gastric atrophy, are also well known.

There has been great progress made on the development of bacterial strain preparations which have widely been used in some medical applications. Some studies have found that probiotics may not only effectively inhibit Helicobacter pylori (H. pylori) infection in vitro, but significantly decrease the colonization by Helicobacter pylori (H. pylori) in vivo and inhibit the inflammatory process in the meantime.

Although some probiotic strains seem to help eradicate Helicobacter pylori (H. pylori) infection, there is still a need to find other more suitable probiotic strains to treat and or prevent the conditions associated with colonization by Helicobacter pylori (H. pylori) of the gastric mucosa and help eradicate Helicobacter pylori (H. pylori).

### OBJECT OF INVENTION

In order to overcome the need for other more suitable bacterial strains for the treatment and or prevention of the conditions associated with the colonization by Helicobacter pylori (H. pylori), the present inventors have studied different probiotic strains, or combinations of strains, with excellent stability when passing through the gastrointestinal tract and their ability to adhere to digestive mucosa.

It is therefore an object of the present invention to provide bacterial strains as described in the present invention, a method, as well as compositions, food products, dietary supplements, or pharmaceutically acceptable formulations comprising such bacterial strains, to be used in treating and/or preventing conditions associated with the colonization by Helicobacter pylori (H. pylori) of the gastric mucosa, in inhibiting Helicobacter pylori (H. pylori), in treating and or preventing Helicobacter pylori (H. pylori) infection, in maintaining gastric microbiota homeostasis and in treating and preventing gastric inflammation in a subject.

### SUMMARY OF THE INVENTION

The object of the present invention is to show the aptitude of some bacterial strains to treat and or prevent conditions associated with colonization by Helicobacter pylori (H. pylori) of the gastric mucosa in a subject.

Another object of the present invention is to show the aptitude of some bacterial strains to inhibit Helicobacter pylori (H. pylori) in a subject.

Another object of the present invention is to show the aptitude of some bacterial strains to treat and or prevent Helicobacter pylori (H. pylori) infection in a subject.

Another object of the present invention is to show the aptitude of some bacterial strains to maintain gastric microbiota homeostasis in a subject.

Another object of the present invention is to show the aptitude of some bacterial strains to treat and prevent gastric inflammation in a subject.

Accordingly, in one aspect, the present invention provides bacterial strain(s) for use in treating and or preventing conditions associated with colonization by Helicobacter pylori (H. pylori) of the gastric mucosa in a subject, wherein said bacterial strain(s) is selected from the strains Lactobacillus acidophilus NCFM (NCFM) and Lactiplantibacillus plantarum Lp-115 (Lp-115), or a combination thereof.

In another aspect, the present invention provides bacterial strain(s) for use in inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

In another aspect, the present invention provides bacterial strain(s) for use in treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

In another aspect, the present invention provides bacterial strain(s) for use in maintaining gastric microbiota homeostasis in a subject, wherein said bacterial strain(s) is selected from the strains Lactobacillus acidophilus NCFM and Lactiplantibacillus plantarum Lp-115, or a combination thereof.

In another aspect, the present invention provides bacterial strain(s) for use in treating and or preventing gastric inflammation in a subject, wherein said bacterial strain(s) is selected from the strains Lactobacillus acidophilus NCFM and Lactiplantibacillus plantarum Lp-115, or a combination thereof.

In a further aspect, the present invention provides a method of treating and or preventing conditions associated with colonization by Helicobacter pylori (H. pylori) of the gastric mucosa in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In yet a further aspect, the present invention provides a method of inhibiting Helicobacter pylori (H. pylori) in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of treating and or preventing Helicobacter pylori (H. pylori) infection in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of maintaining gastric microbiota homeostasis in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In a further aspect, the present invention provides a method of treating and or preventing gastric inflammation in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In a further aspect, the present invention provides a use of bacterial strain(s) for treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

In another aspect, the present invention provides a use of bacterial strain(s) for inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

In a further aspect, the present invention provides a use of bacterial strain(s) for treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

In yet a further aspect, the present invention provides a use of Bacterial strain(s) for maintaining gastric microbiota homeostasis in a subject, wherein said bacterial strain(s) is selected from the strains Lactobacillus acidophilus NCFM and Lactiplantibacillus plantarum Lp-115, or a combination thereof.

In another aspect, the present invention provides a use of Bacterial strain(s) for treating and or preventing gastric inflammation in a subject, wherein said bacterial strain(s) is selected from the strains Lactobacillus acidophilus NCFM and Lactiplantibacillus plantarum Lp-115, or a combination thereof.

### DESCRIPTION OF DRAWINGS

FIG. 1. Six probiotic strains inhibit the adhesion of Helicobacter pylori (H. pylori) on AGS cell: (a) fluorescence microscope images showing the colonization by Helicobacter pylori (H. pylori) P12-GFP on AGS cell upon intervention of six probiotic strains (Lactobacillus acidophilus NCFM, Lactobacillus acidophilus La-14, Lactiplantibacillus plantarum Lp-115, Lacticaseibacillus paracasei Lpc-37, Lacticaseibacillus rhamnosus Lr-32 and Lacticaseibacillus rhamnosus GG); (b) urease activity assay shows interference with the colonization of Helicobacter pylori (H. pylori) P12-GFP on AGS cell in 1-7 hours by the six tested probiotic strains. *(P<0.05);**(P<0.01), compared to H. pylori. ns, not significant.

FIG. 2. Inhibitory effects of six probiotic strains on Helicobacter pylori (H. pylori)-induced inflammation in AGS cell line. AGS cells were co-infected with six probiotic strains (Lactobacillus acidophilus NCFM, Lactobacillus acidophilus La-14, Lactiplantibacillus plantarum Lp-115, Lacticaseibacillus paracasei Lpc-37, Lacticaseibacillus rhamnosus Lr-32 and Lacticaseibacillus rhamnosus GG) and Helicobacter pylori (H. pylori) P12 at a multiplicity of infection (MOI) 100 for 6 h. FIG. 2 shows as follows: (a) and (b) respectively, the mRNA levels of Cxcl8 and TNF in the cells and (c) and (d), respectively, the protein concentrations of IL-8 and TNF-a in the supernatant. The results of each experiment are shown as the mean t standard deviation of three independent experiments. *(P<0.05); **(P<0.01); ***(P<0.001); ****(P<0.0001).

FIG. 3. L. acidophilus NCFM and L. plantarum Lp-115 suppress Helicobacter pylori (H. pylori) adhesion in mice: (a) Mice were fed with Helicobacter pylori (H. pylori) SS1 only or with L. acidophilus NCFM and/or L. plantarum Lp-115 for 6 weeks; (b) The colonization by Helicobacter pylori (H. pylori) was identified by immunohistochemistry (IHC). Uninfected group 200× (A) and 400× (B); Helicobacter pylori (H. pylori) group 200× (C) and 400× (D); Helicobacter pylori (H. pylori)+NCFM group 200× (E) and 400× (F); Helicobacter pylori (H. pylori)+Lp-115 group 200× (G) and 400× (H); Helicobacter pylori (H. pylori)+NCFM+Lp-115 group 200× (I) and 400× (J); (c) The colonization by Helicobacter pylori (H. pylori) was identified by expression of Ure-A and Ure-B by qRT-PCR. Mice were coinfected with Helicobacter pylori (H. pylori) SS1 and Lactobacillus acidophilus NCFM and/or Lactiplantibacillus plantarum Lp-115. Each experiment result shows the mean t standard deviation of three independent experiments. *p<0.05; **(P<0.01); ***(P<0.001).

FIG. 4. L. acidophilus NCFM and L. plantarum Lp-115 suppress Helicobacter pylori (H. pylori) inflammation in mice. (a) The inflammation by Helicobacter pylori (H. pylori) was identified by hematoxylin and eosin staining HE. Uninfected group 100× (A) and 200× (B); Helicobacter pylori (H. pylori) group 100× (C) and 200× (D); Helicobacter pylori (H. pylori)+NCFM group 100× (E) and 200× (F); Helicobacter pylori (H. pylori)+Lp-115 group 100× (G) and 200× (H); Helicobacter pylori (H. pylori)+NCFM+Lp-115 group 100× (I) and 200× (J); (b) The m-RNA expression level of IFN-γ, CXCL15, IL-4, IL-10 in each group. ns(P≥0.05); *(P<0.05); **(P<0.01); ***(P<0.001); ****(P<0.0001).

FIG. 5. Rapid urease test assay for mice model. Clear samples are negative and darker samples are positive. The results show that all samples in the Helicobacter pylori (H. pylori) group were tested positive, and some individual samples in the Lactobacillus and Lactiplantibacillus intervention group were tested negative.

### ADVANTAGES

Although the mechanism of action of the probiotics in the treatment and prevention of the conditions associated with the colonization by Helicobacter pylori (H. pylori) of the gastric mucosa is not fully understood, it was surprisingly found that the ability of the probiotics to adhere to the gastric mucosa and its interference with the adhesion of Helicobacter pylori (H. pylori), with the consequent down-regulation of the expression of the immuno-inflammatory mediators, seems to be the mechanism of action.

Thus, it has surprisingly been found by the present inventors that Lactobacillus acidophilus NCFM and Lactiplantibacillus plantarum Lp-115 can help treat or prevent conditions associated with the colonization by Helicobacter pylori (H. pylori) in a subject by down regulating local Th1 immune response in gastric mucosa, inhibiting pro-inflammatory factor IFN-γ, while promoting Th2 response to produce anti-inflammatory factor IL-4.

Furthermore, our study also shows that Lactobacillus acidophilus NCFM and Lactiplantibacillus plantarum Lp-115 can inhibit and maintain gastric microbiota homeostasis in a subject

### DETAILED DESCRIPTION OF INVENTION

The detailed aspects of this invention are set out below. In part some of the detailed aspects are discussed in separate sections. This is for ease of reference and is in no way limiting. All of the embodiments described below are equally applicable to all aspects of the present invention unless the context specifically dictates otherwise.

### Bacteria

The bacterial strains of the present invention are selected from bacterial strains of the genus Lactobacillus and Lactiplantibacillus. Preferably the bacterial strains of the genus Lactobacillus are of the species Lactobacillus acidophilus, Lacticaseibacillus paracasei and Lacticaseibacillus rhamnosus. Preferably the bacterial strains of the genus Lactiplantibacillus is of the species Lactiplantibacillus plantarum. In particular, the bacterial strains are Lactobacillus acidophilus NCFM, Lactobacillus acidophilus La-14, Lacticaseibacillus paracasei Lpc-37, Lacticaseibacillus rhamnosus Lr-32, Lacticaseibacillus rhamnosus GG and Lactiplantibacillus plantarum Lp-115.

The bacterial strains are commercially available from International Flavors & Fragrances, Inc. (Previously DuPont Nutrition Biosciences Aps of Denmark).

The bacterial strains were also deposited by DuPont Nutrition Biosciences Aps of Denmark, in accordance with the Budapest Treaty on the international recognition of the deposit of microorganisms for the purposes of patent procedure at the Leibniz-Institut, Deutsche Sammlung von Mikroorganismen und Zellkulturen GmbH (DSMZ), Inhoffenstrasse 7B, 38124 Braunschweig, Germany, where they are recorded under the following registration numbers:
1. Strain NCFM (DGCC8698); deposited on 15 Mar. 2021 under the registration number DSM33840 and an accompanying DSMZ deposit form is on record with this specification and incorporated by reference herein.
2. Strain La-14 (DGCC11491); deposited on 1 Jun. 2021 under the registration number DSM33880 and an accompanying DSMZ deposit form is on record with this specification and incorporated by reference herein.
3. Strain Lpc-37 (DGCC4981); deposited on 5 Oct. 2017 under the registration number DSM32661 and an accompanying DSMZ deposit form is on record with this specification and incorporated by reference herein.
4. Strain Lr-32 (DGCC9913); deposited on 15 Jan. 2009 under the registration number DSM22193 and an accompanying DSMZ deposit form is on record with this specification and incorporated by reference herein.
5. Strain Lp-115 (DGCC4715); deposited initially on 9 Feb. 2009 under registration number DSM22266; the deposit has been extended according to rule 9.1 of the Budapest Treaty to be available until 9 Feb. 2051 and an accompanying DSMZ deposit form is on record with this specification and incorporated by reference herein.

Bacterial strain L. rhamnosus GG (DGCC12645) is the world's most documented probiotic strain and is commercially available from different sources, including from International Flavors & Fragrances, Inc. (Previously DuPont Nutrition Biosciences Aps of Denmark). It has been described in more than 250 publications.

Preferably the bacterial strains used in the present invention are bacterial strains which are generally recognised as safe (GRAS) and, which are preferably GRAS approved. GRAS is an American Food and Drug Administration (FDA) designation that a chemical or substance added to food is considered safe for the intended use by experts, and therefore is exempted from the usual Federal Food, Drug, and Cosmetic Act (FFDCA) food additive tolerance requirements.

In a first aspect, the present invention provides a bacterial strain(s) for use in treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

In another aspect, the present invention provides a bacterial strain(s) for use in inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

In another aspect, the present invention provides a bacterial strain(s) for use in treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

In another aspect, the present invention provides a bacterial strain(s) for use in maintaining gastric microbiota homeostasis in a subject, wherein said bacterial strain(s) is selected from the strains Lactobacillus acidophilus NCFM and Lactiplantibacillus plantarum Lp-115, or a combination thereof.

In another aspect, the present invention provides a bacterial strain(s) for use in treating and preventing gastric inflammation in a subject, wherein said bacterial strain(s) is selected from the strains Lactobacillus acidophilus NCFM and Lactiplantibacillus plantarum Lp-115, or a combination thereof.

In a further aspect, the present invention also provides a method of treating and or preventing conditions associated with colonization by Helicobacter pylori (H. pylori) of the gastric mucosa in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of inhibiting Helicobacter pylori (H. pylori) in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of treating and or preventing Helicobacter pylori (H. pylori) infection in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of maintaining gastric microbiota homeostasis in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In another aspect, the present invention provides a method of treating and or preventing gastric inflammation in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

In yet a further aspect, the present invention provides a use of bacterial strain(s) for treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

In one aspect, the present invention provides a use of bacterial strain(s) for inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

In one aspect, the present invention provides a use of bacterial strain(s) for treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

In another aspect, the present invention provides a use of bacterial strain(s) for maintaining gastric microbiota homeostasis in a subject, wherein said bacterial strain(s) is selected from the strains Lactobacillus acidophilus NCFM and Lactiplantibacillus plantarum Lp-115, or a combination thereof.

In yet a further aspect, the present invention provides a use of bacterial strain(s) for treating and or preventing gastric inflammation in a subject, wherein said bacterial strain(s) is selected from the strains Lactobacillus acidophilus NCFM and Lactiplantibacillus plantarum Lp-115, or a combination thereof.

By bacterial strain(s) it is understood one single strain or more than one strain, for example two strains.

In a particular aspect, the bacterial strain(s) according to the present invention is combined with one or more antibiotics. Examples of antibiotics are amoxicillin, clarithromycin, metronidazole, tetracycline and or Bismuth subsalicylate.

In a particular aspect, the bacterial strain according to the present invention is Lactobacillus acidophilus NCFM.

In another aspect, the bacterial strain is strain Lactiplantibacillus plantarum Lp-115.

The bacterial strain Lactobacillus acidophilus NCFM (also known by NCFM) and the bacterial strain Lactiplantibacillus plantarum Lp-115 (also known by Lp-115) can be used in combination. They can also be used, when combined with each other or when individually, in combination with further strains.

Optionally, the bacterial strains Lactobacillus acidophilus NCFM and Lactiplantibacillus plantarum Lp-115 and, when used in aspects of the invention, other bacterial strains, are probiotic bacteria.

The term “probiotic bacteria” is defined as covering any non-pathogenic bacteria which, when administered live in adequate amounts to a host, confer a health benefit on that host. For classification as a “probiotic”, the bacteria must survive passage through the upper part of the digestive tract of the host. They are non-pathogenic, non-toxic and exercise their beneficial effect on health on the one hand via ecological interactions with the resident microbiota in the digestive tract and, on the other hand, via their ability to influence the host physiology and immune system in a positive manner. Probiotic bacteria, when administered to a host in sufficient numbers, have the ability to progress through the intestine, maintaining viability, exerting their primary effects in the lumen and/or the wall of the host's gastrointestinal tract. They then transiently form part of the resident microbiota and this colonisation (or transient colonisation) allows the probiotic bacteria to exercise a beneficial effect, such as the repression of potentially pathogenic micro-organisms present in the microbiota and interactions with the host in the intestine including the immune system.

Thus, in a particular aspect of the present invention, the bacterial strains are probiotic strains. In a particular aspect, the bacterial strain Lactobacillus acidophilus NCFM and Lactiplantibacillus plantarum Lp-115 are probiotic strains.

### Fibres/Prebiotics/Plant Extracts

In one aspect of the present invention, the bacterial strains are used in combination with one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route, but it can be also applied to other microbially colonized sites. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

Plant extracts refers to products extracted and isolated from plants. Plant extracts are important additives in the industry due to their content in bioactive compounds, such as polyphenols and carotenoids, which have antimicrobial and antioxidant activity.

In one aspect, a prebiotic is a selectively fermentable ingredient that allows specific changes, both in the composition and/or activity in the gastrointestinal microbiota that confers benefits upon host well-being and health.

Suitably, the prebiotic, fibre or plant extract may be used according to the present invention in an amount of 0.01 to 100 g/day, preferably 0.1 to 50 g/day, more preferably 0.5 to 20 g/day. In one embodiment, the prebiotic, fibre or plant extract may be used according to the present invention in an amount of 1 to 10 g/day, preferably 2 to 9 g/day, more preferably 3 to 8 g/day. In another embodiment, the prebiotic, fibre or plant extract may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat, unrefined barley and yacon.

Examples of suitable prebiotics include alginate, xanthan, pectin, locust bean gum (LBG), inulin, guar gum, galacto-oligosaccharide (GOS), fructo-oligosaccharide (FOS), polydextrose (e.g. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, glucooligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, (human) milk oligosaccharides and all forms of resistant starches.

In one aspect, the combination of one or more of the bacterial strains and one or more prebiotics and/or fibres and/or plant extracts according to the present invention exhibits a synergistic effect in certain applications (i.e. an effect which is greater than the additive effect of the bacteria when used separately).

In another aspect according to the present invention, the bacterial strain(s) is used in combination with one or more fibres and/or prebiotics.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, microcrystalline cellulose (MCC) or milk oligosaccharide. In a preferred case, the fibre and/or prebiotic is polydextrose. In another preferred case, the fibres and/or prebiotic is polyphenol.

In a further aspect, the invention relates to a composition, a food product, a food ingredient, a dietary supplements or a pharmaceutical acceptable formulation, comprising bacterial strains according to the present invention or a mixture thereof and optionally one or more fibres and/or prebiotics.

### Compositions

The term “composition” is used in the broad sense to mean the way something is composed, i.e. its general makeup. In aspects of the invention, the compositions may consist essentially of a single strain of the species Lactobacillus acidophilus bacteria. In another aspect of the invention, the compositions may consist essentially of a single strain of the species Lactiplantibacillus plantarum. In a further aspect of the invention, the compositions may comprise strains of the species Lactobacillus acidophilus and of the species Lactiplantibacillus plantarum.

Alternatively, the compositions may comprise other components, such as other bacterial strains, biological and chemical components, active ingredients, metabolites, nutrients, fibres, prebiotics, plant extracts, etc.

In one aspect, the bacterial strains according to the present invention are in the form of a composition for use in treating and or preventing conditions associated with colonization by Helicobacter pylori (H. pylori) of the gastric mucosa in a subject in need thereof.

In another aspect, the bacterial strains according to the present invention are in the form of a composition for use in inhibiting Helicobacter pylori (H. pylori) in a subject in need thereof.

In a further aspect, the bacterial strains according to the present invention are in the form of a composition for use in treating and or preventing Helicobacter pylori (H. pylori) infection in a subject in need thereof.

In another aspect, the bacterial strains according to the present invention are in the form of a composition for use in maintaining gastric microbiota homeostasis in a subject in need thereof.

In another aspect, the bacterial strains according to the present invention are in the form of a composition for use in treating and preventing gastric inflammation in a subject in need thereof.

According to one aspect of the present invention, the composition is a spray-dried, frozen or freeze-dried composition.

According to another aspect of the present invention, the composition comprises a cryoprotectant.

In yet a further aspect of the present invention, the bacterial strain(s) of the species Lactobacillus acidophilus is present in the composition in an amount between 10⁶ and 10¹⁴, e.g. between 10⁸ and 10¹² colony forming units (CFU) per dose, optionally 10¹⁰ CFU per dose.

While it is not a requirement that the compositions comprise any support, diluent or excipient, such a support, diluent or excipient may be added and used in a manner which is familiar to those skilled in the art. Examples of suitable excipients include, but are not limited to, microcrystalline cellulose, rice maltodextrin, silicon dioxide, and magnesium stearate. The compositions of the invention may also comprise cryoprotectant components (for example, glucose, sucrose, lactose, trehalose, sodium ascorbate and/or other suitable cryoprotectants).

The terms “composition” and “formulation” may be used interchangeably.

Compositions used in aspects of the invention may take the form of solid, liquid, solution or suspension preparations. Examples of solid preparations include, but are not limited to tablets, pills, capsules, granules, and powders which may be wettable, spray-dried or freeze dried/lyophilized.

The compositions may contain flavouring or colouring agents. The compositions may be formulated for immediate-, delayed-, modified-, sustained-, pulsed- or controlled-release applications.

By way of example, if the compositions of the present invention are used in a tablet form, the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatine and acacia; lubricating agents such as magnesium stearate, stearic acid, glyceryl behenate and talc may be included.

Examples of other acceptable carriers for use in preparing compositions include, for example, water, salt solutions, alcohol, silicone, waxes, petroleum jelly, vegetable oils, polyethylene glycols, propylene glycol, liposomes, sugars, gelatine, lactose, amylose, magnesium stearate, talc, surfactants, silicic acid, viscous paraffin, perfume oil, fatty acid monoglycerides and diglycerides, hydroxymethylcellulose, polyvinylpyrrolidone, and the like.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

Specific non-limiting examples of compositions which can be used in aspects of the invention are set out below for illustrative purposes. These include, but are not limited to food products, food ingredients, functional foods, dietary supplements, pharmaceutical compositions or formulations and medicaments.

### Food Products

The bacterial strains according to the present invention may take the form of a food product or be part of a food product. Here, the term “food” is used in a broad sense and covers food and drink for humans as well as food and drink for animals (i.e. a feed). Preferably, the food product is suitable for, and designed for, human consumption.

In one aspect, the bacterial strains according to the present invention are in the form of a food product for use in treating and or preventing conditions associated with colonization by Helicobacter pylori (H. pylori) of the gastric mucosa in a subject in need thereof.

In another aspect, the bacterial strains according to the present invention are in the form of a food product for use in inhibiting Helicobacter pylori (H. pylori) in a subject in need thereof.

In a further aspect, the bacterial strains according to the present invention are in the form of a food product for use in treating and or preventing Helicobacter pylori (H. pylori) infection in a subject in need thereof.

In another aspect, the bacterial strains according to the present invention are in the form of a food product for use in maintaining gastric microbiota homeostasis in a subject in need thereof.

In another aspect, the bacterial strains according to the present invention are in the form of a food product for use in treating and preventing gastric inflammation in a subject in need thereof.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the food product of the invention may take the form of one of the following:

A fruit juice; a beverage comprising whey protein: a health or herbal tea, a cocoa drink, a milk drink, a lactic acid bacteria drink, a yoghurt and/or a drinking yoghurt, a cheese, an ice cream, a water ice, a dessert, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, a calcium fortified coffee beverage or a fermented vegetable product such as, but not limited to, kimchi and sauerkraut.

Optionally, where the product is a food product, the bacteria of the present invention should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Ingredients

The bacterial strains according to the present invention may take the form, or be part, of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

The bacterial strains according to the present invention may take the form, or be part, of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

### Dietary Supplements

The bacterial strains according to the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: microorganisms, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

In one aspect, the bacterial strains according to the present invention are in the form of a dietary supplement for use in treating and or preventing conditions associated with colonization by Helicobacter pylori (H. pylori) of the gastric mucosa in a subject in need thereof.

In another aspect, the bacterial strains according to the present invention are in the form of a dietary supplement for use in inhibiting Helicobacter pylori (H. pylori) in a subject in need thereof.

In a further aspect, the bacterial strains according to the present invention are in the form of a dietary supplement for use in treating and or preventing Helicobacter pylori (H. pylori) infection in a subject in need thereof.

In another aspect, the bacterial strains according to the present invention are in the form of a dietary supplement for use in maintaining gastric microbiota homeostasis in a subject in need thereof.

In another aspect, the bacterial strains according to the present invention are in the form of a dietary supplement for use in treating and preventing gastric inflammation in a subject in need thereof.

Dietary supplements may be found in many forms such as tablets, capsules, soft gels, gel caps, liquids, or powders. Some dietary supplements can help ensure an adequate dietary intake of essential nutrients; others may help reduce risk of disease.

### Pharmaceutical Formulations

The bacterial strains according to the present invention may be used as—or in the preparation of pharmaceuticals. Here, the term “pharmaceutical” is used in a broad sense—and covers pharmaceuticals for humans as well as pharmaceuticals for animals (i.e. veterinary applications).

In a preferred aspect, the pharmaceutical is for human use.

The pharmaceutical can be for therapeutic purposes—which may be curative, palliative, alleviating or preventive in nature.

A pharmaceutical may be in the form of a compressed tablet, tablet, powder, capsule, ointment, suppository or drinkable solution.

When used as—or in the preparation of—a pharmaceutical, the bacterial strains of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a liquid or as a solid—depending on the use and/or the mode of application and/or the mode of administration.

The strains of the species Lactobacillus acidophilus and of the species Lactiplantibacillus plantarum used in the present invention may themselves constitute a pharmaceutically active ingredient. In one embodiment, the Lactobacillus acidophilus constitutes the sole active component. In another embodiment Lactiplantibacillus plantarum constitutes the sole active component. In another embodiment, strains of both species Lactobacillus acidophilus and Lactiplantibacillus plantarum are present. Alternatively, the bacterial strains of the present invention may be at least one of a number (i.e. two or more) of pharmaceutically active components, e.g., other bacterial strains.

### Medicaments

The bacterial strains according to the present invention may take the form of medicaments.

The term “medicament” as used herein encompasses medicaments for both human and animal usage in human and veterinary medicine. In addition, the term “medicament” as used herein means any substance which provides a therapeutic, preventive and/or beneficial effect. The term “medicament” as used herein is not necessarily limited to substances which need marketing approval but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Medical Foods

The bacterial strains according to the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Dosage

The bacterial strains according to the present invention may be present in the different forms, i.e., in the compositions, food products, dietary supplements, or pharmaceutically acceptable formulations, in the amount from 10⁶ to 10¹⁴ colony forming units (CFU) of bacterial strain(s) per dose or per gram of composition, food product, dietary supplement, or pharmaceutically acceptable formulation, and more particularly from 10⁸ to 10¹² CFU of bacterial strain(s) per dose or per gram of composition, food product, dietary supplement, or pharmaceutically acceptable formulation.

Optionally the compositions, food products, dietary supplements, or pharmaceutically acceptable formulations comprise about 10¹⁰ CFU of bacterial strain(s) per dose or per gram of the composition, food product, dietary supplement, or pharmaceutically acceptable formulation.

The bacterial strains(s), for example Lactobacillus acidophilus NCFM, and/or Lactiplantibacillus plantarum Lp-115, may be administered at a dosage from about 10⁶ to about 10¹⁴ CFU of bacterial strain per dose, preferably about 10⁸ to about 10¹² CFU of bacterial strain per dose. By the term “per dose” it is meant that this number of bacteria is provided to a subject either per day or per intake, preferably per day. For example, if the bacteria are to be administered in a food product, for example in a yoghurt, then the yoghurt may contain from about 10⁶ to 10¹⁴ CFU of the bacterial strain. Alternatively, however, this number of bacteria may be split into multiple administrations, each consisting of a smaller amount of microbial loading—so long as the overall amount of bacterial strain received by the subject in any specific time, for instance each 24 h period, is from about 10⁶ to about 10¹⁴ CFU of bacteria, optionally 10⁸ to about 10¹² CFU of bacteria.

In one aspect, the bacterial strains of the present invention are present in a therapeutically effective amount in the composition, in the food product, in the dietary supplement, or in the pharmaceutically acceptable formulation.

In accordance with the present invention an effective amount of at least one bacterial strain may be at least 10⁶ CFU of bacteria/dose, optionally from about 10⁸ to about 10¹² CFU of bacteria/dose, e.g., about 10¹⁰ CFU of bacteria/dose.

In one embodiment, the Lactobacillus acidophilus or Lactiplantibacillus plantarum strains, may be administered at a dosage from about 10⁶ to about 10¹⁴ CFU of bacteria/day, optionally about 10⁸ to about 10¹² CFU of bacteria/day. Hence, the effective amount may be from about 10⁶ to about 10¹⁴ CFU of bacteria/day, optionally about 10⁸ to about 10¹² CFU of bacteria/day.

In a particular embodiment, an amount of 1×10⁹ CFU of single bacterial strain or 1.5×10⁹ CFU of bacterial multi-strain were administered.

### Effects/Subjects/Medical Indications

In one embodiment, the term “subject”, as used herein, means a mammal, including for example livestock (for example cattle, horses, pigs, and sheep) and humans. In one embodiment the subject is a human. In one embodiment the subject is female. In one embodiment the subject is male.

In another embodiment, the subject is a dog (such as a member of the genus Canis) or a cat (such as a member of the genera Felis or Panthera). In preferred embodiments, the bacterial strain(s) and compositions, food products, dietary supplements, or pharmaceutically acceptable formulations are for use in a human.

### Other Embodiments of the Invention

Further embodiments of the present invention include, but are not limited to, those set out below: Embodiment 1. Bacterial strain(s) for use in treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

Embodiment 2. Bacterial strain(s) for use in inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

Embodiment 3. Bacterial strain(s) for use in treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

Embodiment 4. Bacterial strain(s) for use in maintaining gastric microbiota homeostasis in a subject, wherein said bacterial strain(s) is selected from the strains Lactobacillus acidophilus NCFM and Lactiplantibacillus plantarum Lp-115, or a combination thereof.

Embodiment 5. Bacterial strain(s) for use in treating and preventing gastric inflammation in a subject, wherein said bacterial strain(s) is selected from the strains Lactobacillus acidophilus NCFM and Lactiplantibacillus plantarum Lp-115, or a combination thereof.

Embodiment 6. The bacterial strain(s) for use according to any one of embodiments 1-5, wherein said bacterial strain(s) is combined with one or more antibiotic.

Embodiment 7. The bacterial strain(s) for use according to embodiment 6, wherein said antibiotics are, but not limited to, amoxicillin, clarithromycin, metronidazole, tetracycline and or Bismuth subsalicylate.

Embodiment 8. The bacterial strain(s) for use according to any one of embodiments 1-7 wherein the strain(s) is Lactobacillus acidophilus NCFM.

Embodiment 9. The bacterial strain(s) for use according to any one of embodiments 1-7 wherein the strain(s) is Lactiplantibacillus plantarum Lp-115.

Embodiment 10. The bacterial strain(s) for use according to any one of embodiments 1-7 wherein strains Lactobacillus acidophilus NCFM and Lactiplantibacillus plantarum Lp-115 are used in combination.

Embodiment 11. The bacterial strain(s) for use according to any one of embodiments 1-10, wherein the bacterial strain(s) is used in combination with one or more fibres and/or prebiotics.

Embodiment 12. The bacterial strain(s) for use according to embodiment 11, wherein the fibres and/or the prebiotic is polydextrose and/or Polyphenol.

Embodiment 13. The bacterial strain(s) for use according to any one of embodiments 1-12, wherein the bacterial strains(s) is in the form of a composition, a food product, a dietary supplement, or a pharmaceutically acceptable formulation.

Embodiment 14. The bacterial strain(s) for use according to embodiment 13, wherein the pharmaceutically acceptable formulation is a medicament.

Embodiment 15. The bacterial strain(s) for use according to embodiment 13, wherein the food product is a medical food product.

Embodiment 16. Method of treating and or preventing conditions associated with colonization by Helicobacter pylori (H. pylori) of the gastric mucosa in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

Embodiment 17. Method of inhibiting Helicobacter pylori (H. pylori) in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

Embodiment 18. Method of treating and or preventing Helicobacter pylori (H. pylori) infection in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

Embodiment 19. Method of maintaining gastric microbiota homeostasis in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

Embodiment 20. Method of treating and or preventing gastric inflammation in a subject in need thereof, said method comprising administering bacterial strain(s) to said subject, wherein said bacterial strain(s) is selected from the strains Lactobacillus acidophilus NCFM and Lactiplantibacillus plantarum Lp-115, or a combination thereof.

Embodiment 21. The method according to any one of embodiments 16-20, wherein said bacterial strain(s) is combined with one or more antibiotics.

Embodiment 22. The method according to embodiment 21, wherein said antibiotics are amoxicillin, clarithromycin, metronidazole, tetracycline and or Bismuth subsalicylate.

Embodiment 23. The method according to any one of embodiments 16-22 wherein the strain is Lactobacillus acidophilus NCFM.

Embodiment 24. The method according to any one of embodiments 16-22 wherein the strain is Lactiplantibacillus plantarum Lp-115.

Embodiment 25. The method according to any one of embodiments 16-22 wherein Lactobacillus acidophilus NCFM and Lactiplantibacillus plantarum Lp-115 are used in combination.

Embodiment 26. The method according to any one of embodiments 16-25, wherein the bacterial strain(s) is used in combination with one or more fibres and/or prebiotics.

Embodiment 27. The method according to embodiment 26, wherein the fibres and/or the prebiotic is polydextrose and/or Polyphenol.

Embodiment 28. The method according to any one of embodiments 16-28, wherein the bacterial strains(s) is the form of a composition, a food product, a dietary supplement, or a pharmaceutically acceptable formulation.

Embodiment 29. The method according to embodiment 28, wherein the pharmaceutically acceptable formulation is a medicament.

Embodiment 30. The method according to embodiment 28, wherein the food product is a medical food product.

Embodiment 31. Use of bacterial strain(s) for treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

Embodiment 32. Use of bacterial strain(s) for inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

Embodiment 33. Use of bacterial strain(s) for treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

Embodiment 34. Use of Bacterial strain(s) for maintaining gastric microbiota homeostasis in a subject, wherein said bacterial strain(s) is selected from the strains Lactobacillus acidophilus NCFM and Lactiplantibacillus plantarum Lp-115, or a combination thereof.

Embodiment 35. Use of Bacterial strain(s) for treating and or preventing gastric inflammation in a subject, wherein said bacterial strain(s) is selected from the strains Lactobacillus acidophilus NCFM and Lactiplantibacillus plantarum Lp-115, or a combination thereof.

Embodiment 36. The use of bacterial strain(s) according to any one of embodiments 31-35, wherein said bacterial strain(s) is combined with one or more antibiotic.

Embodiment 37. The use of bacterial strain(s) according to embodiment 36, wherein said antibiotics are, but not limited to, amoxicillin, clarithromycin, metronidazole, tetracycline and or Bismuth subsalicylate.

Embodiment 38. The use of bacterial strain(s) according to any one of embodiments 31-37 wherein the strain is Lactobacillus acidophilus NCFM.

Embodiment 39. The use of bacterial strain(s) according to any one of embodiments 31-37 wherein the strain is Lactiplantibacillus plantarum Lp-115.

Embodiment 40. The use of bacterial strain(s) according to any one of embodiments 31-37 wherein Lactobacillus acidophilus NCFM and Lactiplantibacillus plantarum Lp-115 are used in combination.

Embodiment 41. The use of bacterial strain(s) according to any one of embodiments 31-40, wherein the bacterial strain(s) is used in combination with one or more fibres and/or prebiotics.

Embodiment 42. The use of bacterial strain(s) according to embodiment 41, wherein the fibres and/or the prebiotic is polydextrose and/or Polyphenol.

Embodiment 43. The use of bacterial strain(s) according to any one of embodiments 31-42, wherein the bacterial strains(s) is in the form of a composition, a food product, a dietary supplement, or a pharmaceutically acceptable formulation.

Embodiment 44. The use of bacterial strain(s) according to embodiment 43, wherein the pharmaceutically acceptable formulation is a medicament.

Embodiment 45. The use of bacterial strain(s) according to embodiment 43, wherein the food product is a medical food product.

### EXAMPLES

The following examples are provided to demonstrate and further illustrate specific embodiments and aspects of the present invention and are not to be construed as limiting the scope thereof.

### Materials and Methods

Bacterial Strains, Cell line and animals

Helicobacter pylori (H. pylori) P12 and H. pylori P12-GFP strains were obtained from Max Planck Institute for Infection Biology, Berlin, Germany (MPIIB) and Helicobacter pylori (H. pylori) SS1 was obtained from University of Western Australia (UWA), Australia.

Helicobacter pylori (H. pylori) strains were cultured on Columbia medium plate containing 7% sterile defibrillated sheep blood (Bianzhen, Nanjiing), 20 μg/ml vancomycin (Meilunbio), 10 μg/ml polymyxin (Meilunbio), 10 μg/ml amphotericin B (Meilunbio), 10 μg/ml trimethoprim (Sigma), then placed in an incubator containing 5% 02 and 10% CO₂, cultured at 37° C., subcultured once every 3 days, and used for experiment after subculture.

Lactobacillus, Lacticaseibacillus and Lactiplantibacillus strains were provided by Danisco (China) Holding Co., Ltd., Lactobacillus acidophilus NCFM, Lactobacillus acidophilus La-14 (La-14), Lactiplantibacillus plantarum Lp-115 (Lp-115), Lacticaseibacillus paracasei Lpc-37 (Lpc-37), Lacticaseibacillus rhamnosus Lr-32 (Lr-32) and Lacticaseibacillus rhamnosus GG. After the Gram Staining Kit (Solarbio) was used to identify the bacterial morphology, the strains were anaerobic cultured in MRS broth medium (Solarbio) at 37° C. for 48 h and then subcultured.

Human gastric adenocarcinoma cell line (AGS) was purchased from the Institute of Biochemistry and Cell Biology of the Chinese Academy of Sciences (Shanghai, China). AGS cells were cultured in RPMI 1640 medium (Thermo) supplemented with 10% fetal bovine serum at 37° C. and 5% CO2 in a humidified incubator.

Fifty male C57BL/6 mice (Specific pathogen Free, 4 weeks old) were purchased from Zhejiang Vital River Laboratory Animal Technology Co. Ltd. All animals were housed under standard conditions

Helicobacter pylori (H. pylori) and Lactobacillus co-infection model in vitro

Helicobacter pylori P12 and lactobacilli were cultured overnight in BHI (Thermo Fisher) and MRS broth. After centrifugation at 5000× rpm for 8 min and 4000× rpm for 5 min, the supernatant was discarded, and bacteria were harvested and resuspended in 1 ml serum-free RPMI 1640 medium.

Overnight cultured AGS cells were co-infected with lactobacilli (MOI=100) and Helicobacter pylori(H. pylori) P12 (MOI=100) for 6 h. After the incubation period, the supernatant was harvested for ELISA and cells were harvested for RNA isolation.

Adhesion of Helicobacter pylori (H. pylori) on AGS cell

AGS cells were cultured in two 12 well plates and incubated with the Lactobacilli (MOI=100) and Helicobacter pylori (H. pylori) P12-GFP (MOI=100) for 6 h. At the end of incubation, one plate of the cells was washed 3 times with PBS and photographed by fluorescence and white light. Configuration of urease activity assay reagent before end of infection: Add concentrated sodium hydroxide into the PBS (PH=7.4) to adjust the solution (PH=6.8), and urea (Solarbio) and phenol red (Solarbio) were weighed and added to reach concentrations of urea 110 mmol/L and phenol red 10 mg/L respectively, and then dissolved by vigorous shaking. To the other 12 well plates 1 ml urease detection reagent was added. After reaction for 1-7 h, 100 μl medium was withdrawn to 96 well plate to record the absorbance value at 540 nm.

Quantitative real-time PCR (qRT-PCR)

AGS cells and mouse gastric mucosal tissue were lysed with RNAiso plus (TaKaRa, Japan). cDNA was converted using ReverTra Ace qPCR RT Kit (TOYOBO, Shanghai) according to the manufacturer's instruction. qRT-PCR was performed using 2×ChamQ Universal SYBR qPCR Master Mix (Vazyme, Nanjing, China) in a Roche Lightcycler480II system based on the manufacturer's recommendations. Primers sequences were designed in NCBI Primer-BLAST (Table S1). The relative gene expression was determined using the 2^{-ΔΔCt} method. All experiments were repeated three times.

TABLE S1
Primers for the quantification of inflammatory factors in AGS cells
and mouse gastric tissue

| Species | Gene | Primer | Nucleotide Sequence (5′-3′) |
|---|---|---|---|
| Human | TNF | Forward | CCCAGGGACCTCTCTCTAATCA (SEQ ID NO: 1) |
| | TNF | Reverse | GCTACAGGCTTGTCACTCGG (SEQ ID NO: 2) |
| | Cxcl8 | Forward | ACTGAGAGTGATTGAGAGTGGAC (SEQ ID NO: 3) |
| | Cxcl8 | Reverse | AACCCTCTGCACCCAGTTTTC (SEQ ID NO: 4) |
| | GAPDH | Forward | GGTATCGTGGAAGGACTCATGAC (SEQ ID NO: 5) |
| | GAPDH | Reverse | ATGCCAGTGAGCTTCCCGTTCAG (SEQ ID NO: 6) |
| Mouse | ureA | Forward | GCTGGTGCGATTGGCTTTA (SEQ ID NO: 7) |
| | ureA | Reverse | GGATAGCGACTTGCACATCGT (SEQ ID NO: 8) |
| | ureB | Forward | GCCCACTTCTACAGAACCGACATAC (SEQ ID NO: 9) |
| | ureB | Reverse | AGGCGATAACGACAACTTCAGGATC (SEQ ID NO: 10) |
| | Il10 | Forward | CCAGGGAGATCCTTTGATGA (SEQ ID NO: 11) |
| | Il10 | Reverse | AACTGGCCACAGTTTTCAGG (SEQ ID NO: 12) |
| | Il4 | Forward | GGTCTCAACCCCCAGCTAGT (SEQ ID NO: 13) |
| | Il4 | Reverse | GCCGATGATCTCTCTCAAGTGAT (SEQ ID NO: 14) |
| | Ifng | Forward | CAGGCCATCAGCAACAACATAAGC (SEQ ID NO: 15) |
| | Ifng | Reverse | AGCTGGTGGACCACTCGGATG (SEQ ID NO: 16) |
| | Cxcl15 | Forward | CGGCAATGAAGCTTCTGTAT (SEQ ID NO: 17) |
| | Cxcl15 | Reverse | CCTTGAAACTCTTTGCCTCA (SEQ ID NO: 18) |
| | GAPDH | Forward | GCTGAGTATGTCGTGGAG (SEQ ID NO: 19) |
| | GAPDH | Reverse | TCTTCTGAGTGGCAGTGAT (SEQ ID NO: 20) |

### Cytokine Profiles Quantification by ELISA

After infection, collect the final culture medium, centrifuge at 12000 rpm for 5 min, remove cell debris and bacteria, and collect the supernatant. The blood of mice was collected from the orbital venous plexus of mice in each group, and incubated overnight at 4° C., and then the samples were centrifuged at 3500 rpm for 20 min to collect the supernatant. The concentration of IL-8 and TNF-a were detected by human IL-8 (Interleukin 8) ELISA Kit (Elabscience, WH, China) and human TNF-a (Tumor necrosis factor alpha) ELISA Kit (Elabscience, WH, China) respectively. Mice serum levels of IL-4, IL-10, and IFN-γ were measured by V-PLEX Proinflammatory Panel 1 (mouse) Kit (Meso Scale Discovery, Rockville, MD, USA). The operation was carried out according to the specific steps in the manual. All experiments were repeated 3 times.

Establish the model of Helicobacter pylori (H. pylori) infection and bacterial strains Lactobacillus and Lactiplantibacillus intervention in mice.

Specific pathogen-free C57BL/6 mice (male; 4 weeks old) were obtained from Zhejiang Vital River Laboratory Animal Technology Co., Ltd., and fed on the Laboratory Rodent Diet 5001. 50 mice were randomly divided into 5 groups after adaptive feeding for 1 week:
Uninfected;
Helicobacter pylori (H. pylori) SS1;
Helicobacter pylori (H. pylori) SS1+L. acidophilus NCFM;
Helicobacter pylori (H. pylori) SS1+Lactiplantibacillus plantarum Lp-115;
Helicobacter pylori (H. pylori) SS1+L. acidophilus NCFM+Lactiplantibacillus plantarum Lp-115.

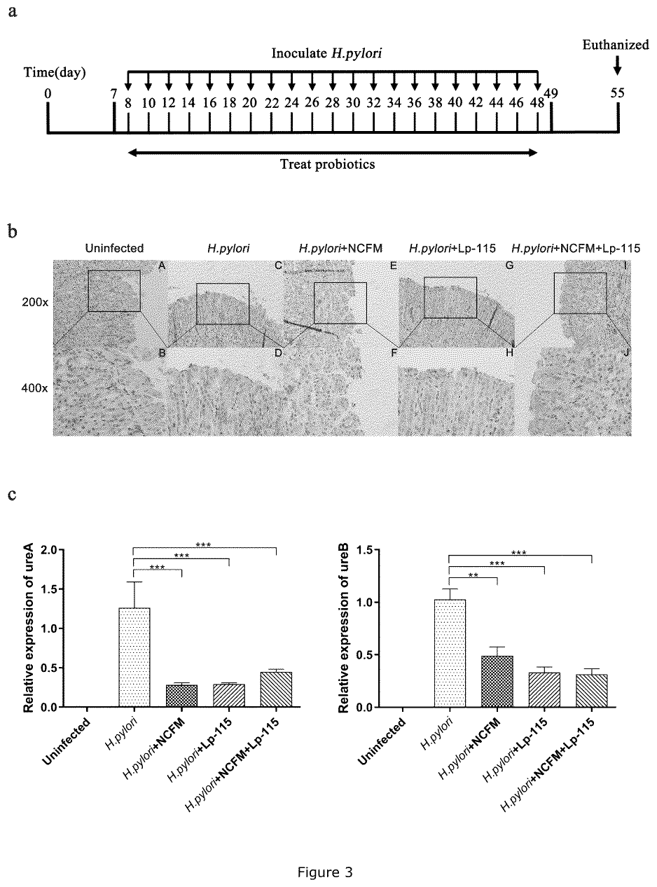

The control group was gavaged with PBS, and the experimental groups were gavaged with Helicobacter pylori (H. pylori) SS1 (1*10⁹ CFU/ml) only or combined with the corresponding bacterial strain (1*10⁹ CFU/ml) once on 2 days for 6 weeks (FIG. 3a).

Rapid urease test (RUT)

Take the gastric mucosa of mice and use the rapid urease Kit (Sanqiang, FJ, China) for rapid urease test. The colour change was determined. When the solution turns pink or red, the urease test is positive; when it is still yellow, the urease test is negative.

### Hematoxylin-Eosin (HE) Staining and Immunohistochemistry (IHC)

Gastric tissues were fixed with 4% paraformaldehyde, embedded in paraffin, cut into 4 mm sections, and then stained by HE method and immunohistochemical method, respectively. Chronic gastritis histological grade scale: there are five histological changes to be graded: Helicobacter pylori, chronic inflammation, activity, atrophy, and intestinal metaplasia, each classified into four grades: none, mild, moderate and severe. According to the new Sydney system, the degree of inflammation and lymphocyte infiltration of gastric tissue after Helicobacter pylori infection were evaluated. Immunohistochemistry for Helicobacter pylori (H. pylori) was performed using a Rabbit anti Helicobacter pylori (H. pylori) polyclonal antibody (Cell Marque) (ZSGB, BJ, China), identifying Helicobacter pylori (H. pylori) colonization of the mouse gastric mucosa.

### Statistics

All statistical analyses were performed using GraphPad Prism 9 software. During the processing of experimental data, the values whose deviation from the average value of the same group of data exceeded three times the standard deviation were considered outliers and eliminated. We performed one-way ANOVA on raw and Ig-converted data to compare the multi groups. The Bonferroni and Tukey tests were conducted to calculate the statistical significance among the groups. P-value <0.05 was considered significant for all statistical analyses.

### Results

Six Lactobacillus and Lactiplantibacillus strains inhibit the adhesion of Helicobacter pylori on AGS cell.

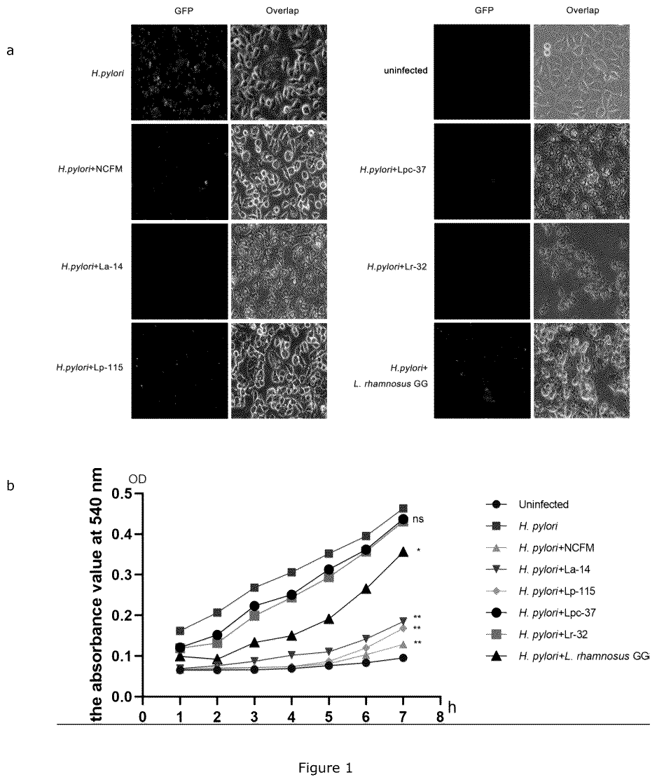

To compare the effect of six probiotic strains in intervening Helicobacter pylori (H. pylori) adhesion, AGS cells were co-infected with Helicobacter pylori (H. pylori) P12-GFP and six probiotic bacterial strains (MOI=100) respectively for 6 hours. After infection, cells and GFP-positive Helicobacter pylori (H. pylori) were measured under fluorescence microscopy. The number of GFP-positive Helicobacter pylori (H. pylori) in the six probiotic strains intervening group was less than that in the Helicobacter pylori (H. pylori) sole infected group. Furthermore, L. acidophilus NCFM and L. plantarum Lp-115 groups have less cell stress on the AGS cell compared with other Lactobacillus intervening groups (FIG. 1a).

To further quantify the inhibition effect between different probiotic strains, we used urease activity assay to deal with the co-infection plate as mentioned above and record the absorbance value at 540 nm. The absorbance value of the six probiotic strains intervention group was decreased to different degrees compared with that of the Helicobacter pylori (H. pylori) sole infected group from 1-7 h (FIG. 1b). Thus, it proved that these six probiotic strains can effectively reduce the colonization by Helicobacter pylori (H. pylori), and furthermore, L. acidophilus NCFM and L. plantarum Lp-115 have less cell harm to AGS cell.

Six Probiotic Strains Inhibit the Helicobacter pylori Induced Inflammation on AGS Cell.

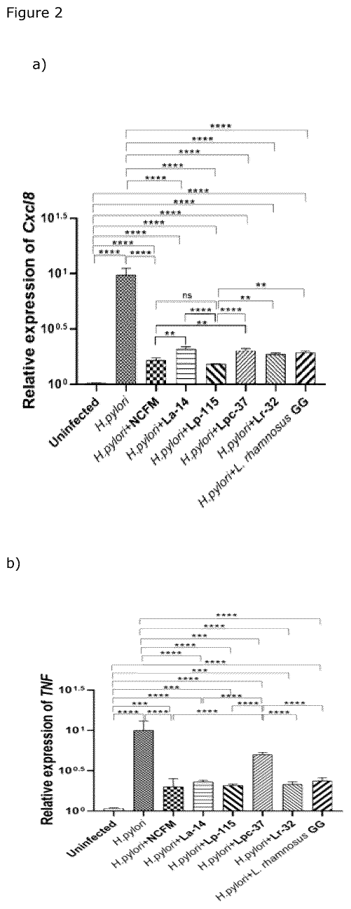

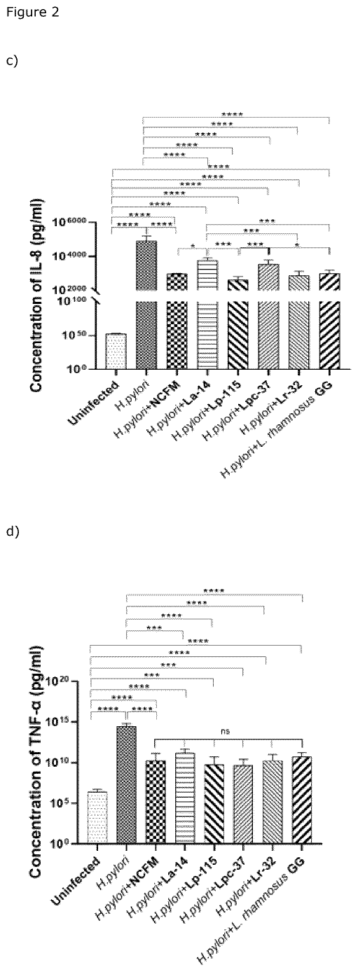

Helicobacter pylori (H. pylori) infection induces the inflammation of gastric mucosa and activate the cytokines such as IL-8 and TNF-a. To compare the inhibition effect between the six probiotic strains, we performed the co-infection model of Helicobacter pylori (H. pylori) and six Lactobacillus strains (MOI=100) in AGS cell for 6 h and check the mRNA and protein level of IL-8 and TNF-a. It is shown that the mRNA expression of IL-8 and TNF-a in the six probiotic strains intervention groups were significantly decreased compared with those in the Helicobacter pylori (H. pylori) sole infected group (FIGS. 2(a) and 2(b).

Consistently, the contents of IL-8 and TNF-a in the cell supernatant were also significantly decreased in the probiotic strain intervention groups compared with the Helicobacter pylori (H. pylori) sole infected group (FIGS. 2(b) and 2(c). Combine the mRNA and protein level data, it shown that L. acidophilus NCFM, and L. plantarum Lp-115 have better intervention effect compared with other stains which need to be further validated in the Helicobacter pylori (H. pylori) infected mouse model.

Lactobacillus acidophilus NCFM and Lactiplantibacillus Plantarum Lp-115 Suppress Helicobacter pylori Colonization in Mice.

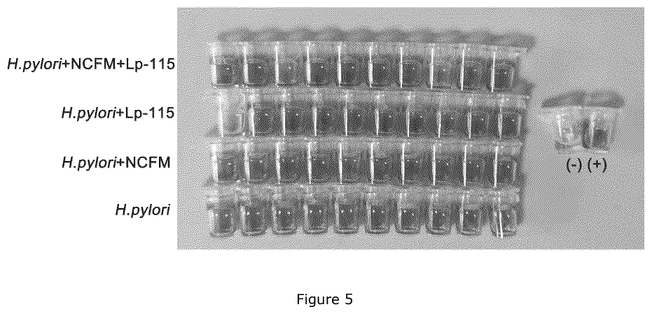

To further validate whether L. acidophilus NCFM and L. plantarum Lp-115 can attenuate Helicobacter pylori (H. pylori) colonization and gastric inflammation in vivo, C57BL/6 mice were coinfected with L. acidophilus NCFM and L. plantarum Lp-115 with Helicobacter pylori (H. pylori) SS1 for 6 weeks (FIG. 3a). After infection, mice were euthanized, and the gastric tissues were assessed for Helicobacter pylori (H. pylori) infection by the rapid urease test (FIG. 5). The Helicobacter pylori (H. pylori) adhesion on gastric tissues were then analyzed by immunohistochemistry (IHC) assays (FIG. 3b), which shown that L. acidophilus NCFM and/or L. plantarum Lp-115 intervention groups have comparable less Helicobacter pylori (H. pylori) adhesion than the Helicobacter pylori (H. pylori) sole infected group. To further validate the Helicobacter pylori (H. pylori) colonization in different group, the mRNA expression of Ure-A and Ure-B were tested from gastric tissues of all groups (FIG. 3c), which shown that L. acidophilus NCFM and/or L. plantarum Lp115 intervention groups have less expression of Ure-A and Ure-B compared with Helicobacter pylori (H. pylori) sole infected group. This further proved that L. acidophilus NCFM and/or L. plantarum Lp-115 can inhibit the Helicobacter pylori (H. pylori) colonization on gastric mucosa in vivo.

Lactobacillus acidophilus NCFM and Lactiplantibacillus Plantarum Lp-115 Suppress Helicobacter pylori Induced Inflammation in Mice.

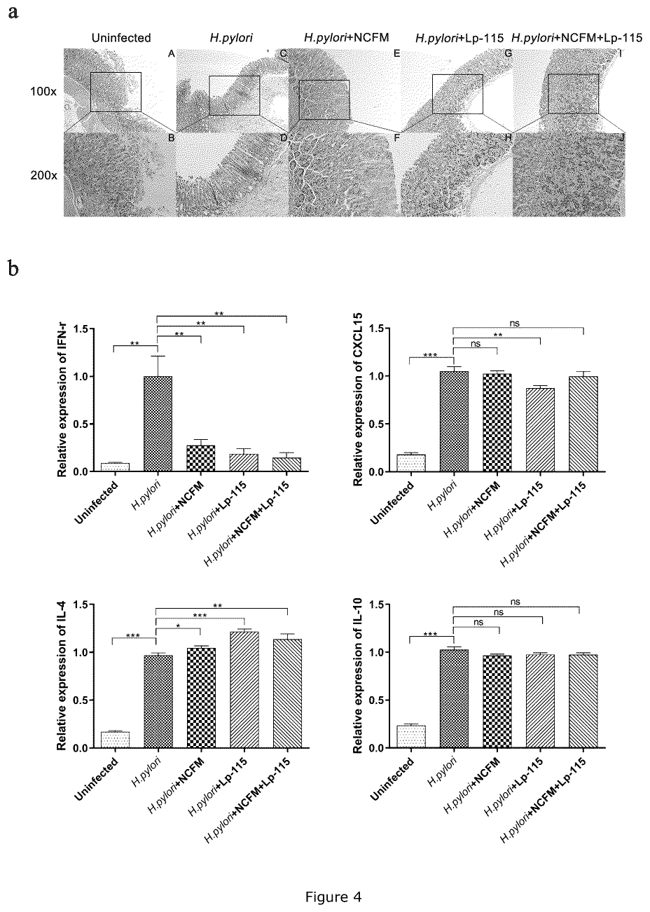

To further investigate whether L. acidophilus NCFM and L. plantarum Lp-115 can attenuate Helicobacter pylori (H. pylori) colonization and gastric inflammation in vivo, gastric tissues from 5 groups were then analyzed by Hematoxylin-Eosin staining (HE stain). Compared with the uninfected group, the gastric lamina propria of most mice in the Helicobacter pylori (H. pylori) infected group showed more lymphocyte and plasma cell infiltration and neutrophil infiltration in the active phase, and some mice also had local thinning, reduction of the glands propria, and thickening of the muscularis mucosae; Compared with the Helicobacter pylori (H. pylori) group, the inflammatory cells in the infiltrating the lamina propria of the gastric mucosa in the Helicobacter pylori (H. pylori)+NCFM group, Helicobacter pylori (H. pylori)+Lp-115 group and Helicobacter pylori (H. pylori)+NCFM+Lp-115 group were reduced to different degrees, which indicated that L. acidophilus NCFM and L. plantarum Lp-115 could ameliorate the Helicobacter pylori (H. pylori) induced gastric inflammation (FIG. 4a).

To further verify if strains NCFM and/or Lp-115 have the ability to inhibit Helicobacter pylori (H. pylori) induced Th1 inflammation, the expression of IL-4, CXCL15, IL-10, IFN-γ was detected by qRT-PCR. It showed that Helicobacter pylori (H. pylori) infection significantly increases the expression of IL-4, CXCL15, IL-10, IFN-γ, While after strains NCFM and Lp-115 intervention, there was a variable reduction of IFN-γ in each intervention group and slightly reduction of CXCL15 in Lp-115 intervention group; Moreover, the expression of IL-4 was elevated in different degrees, in Lp-115 and NCFM+Lp-115 intervention group. The mRNA expression of IL-10 in the NCFM and Lp-115 intervention group did not change compared with those in the Helicobacter pylori (H. pylori) group (FIG. 4b). In all, it showed that NCFM and/or Lp-115 can inhibit Helicobacter pylori (H. pylori) induced Th1 type inflammation in C57BL/6 mice and activate the Th2 inflammation.

### Discussion

In our study, we screened 6 probiotic strains and found that L. acidophilus NCFM and/or L. plantarum Lp-115 can inhibit Helicobacter pylori (H. pylori) adhesion through in vitro AGS cell line and in vivo C57/BL6 mouse model, which is the first validation study of these two strains. In this study, it is shown for the first time that L. acidophilus NCFM and/or L. plantarum Lp-115 can inhibit Helicobacter pylori (H. pylori) P12 induced IL-8 and TNF-a expression in mRNA and protein level in AGS cell line. In Helicobacter pylori (H. pylori) SS1 infected C57BL/6 mouse model, the present study also confirmed that L. acidophilus NCFM and/or L. plantarum Lp-115 can inhibit IFN-γ but increase IL-4.

In this study, the intervention of L. acidophilus NCFM and L. plantarum Lp-115 are shown to be able to alleviate Helicobacter pylori (H. pylori) infection induced host inflammatory response by down regulating local Th1 immune response in gastric mucosa, inhibiting proinflammatory factor IFN-γ, while promoting Th2 response to produce anti-inflammatory factor IL-4. Thus, L. acidophilus NCFM and L. plantarum Lp-115 may play an important role in promoting the differentiation of T cells into Th2 cells to balance Hp induced Th1 inflammation.

In the present study it is also shown that some Lactobacilli strains are harmful from AGS cell model, including Lacticaseibacillus rhamnosus Lr-32, Lacticaseibacillus paracasei Lpc-37, Lacticaseibacillus rhamnosus GG and Lactobacillus acidophilus La-14, while L. acidophilus NCFM and L. plantarum Lp-115 are safe.

In conclusion, the present study has shown that strain NCFM and strain Lp-115 have the effect to inhibit Helicobacter pylori (H. pylori) adhesion and host inflammatory responses in cell line and mouse model and have therefore shown a unique value in the treatment of Helicobacter pylori (H. pylori), such as treat and or prevent conditions associated with colonization by Helicobacter pylori (H. pylori) of the gastric mucosa, treat and or prevent Helicobacter pylori (H. pylori) infection, maintain gastric microbiota homeostasis and treat and or prevent gastric inflammation in a subject.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. Bacterial strain(s) for use in treating and or preventing conditions associated with colonization by Helicobacter pylori (H. pylori) of the gastric mucosa in a subject, wherein said bacterial strain(s) is selected from the strains Lactobacillus acidophilus NCFM and Lactiplantibacillus plantarum Lp-115, or a combination thereof.

2. Bacterial strain(s) for use in inhibiting Helicobacter pylori (H. pylori) in a subject, wherein said bacterial strain(s) is selected from the strains Lactobacillus acidophilus NCFM and Lactiplantibacillus plantarum Lp-115, or a combination thereof.

3. Bacterial strain(s) for use in treating and or preventing Helicobacter pylori (H. pylori) infection in a subject, wherein said bacterial strain(s) is selected from the strains Lactobacillus acidophilus NCFM and Lactiplantibacillus plantarum Lp-115, or a combination thereof.

4. Bacterial strain(s) for use in maintaining gastric microbiota homeostasis in a subject, wherein said bacterial strain(s) is selected from the strains Lactobacillus acidophilus NCFM and Lactiplantibacillus plantarum Lp-115, or a combination thereof.

5. Bacterial strain(s) for use in treating and preventing gastric inflammation in a subject, wherein said bacterial strain(s) is selected from the strains Lactobacillus acidophilus NCFM and Lactiplantibacillus plantarum Lp-115, or a combination thereof.

6. The bacterial strain(s) for use according to claim 1, wherein said bacterial strain(s) is combined with one or more antibiotics.

7. The bacterial strain(s) for use according to claim 6, wherein said antibiotics are, but not limited to, amoxicillin, clarithromycin, metronidazole, tetracycline and Bismuth subsalicylate or Bismuth subsalicylate.

8. The bacterial strain(s) for use according to claim 1, wherein the strain(s) is Lactobacillus acidophilus NCFM.

9. The bacterial strain(s) for use according to claim 1, wherein the strain(s) is Lactiplantibacillus plantarum Lp-115.

10. The bacterial strain(s) for use according to claim 1, wherein strains Lactobacillus acidophilus NCFM and Lactiplantibacillus plantarum Lp-115 are used in combination.

11. The bacterial strain(s) for use according to claim 1, wherein the bacterial strain(s) is used in combination with one or more fibers or prebiotics or a combination of any of the foregoing.

12. The bacterial strain(s) for use according to claim 11, wherein the fibers and the prebiotic or the prebiotic is polydextrose and polyphenol or polyphenol.

13. The bacterial strain(s) for use according to claim 1, wherein the bacterial strains(s) is in the form of a composition, a food product, a dietary supplement, or a pharmaceutically acceptable formulation.

14. The bacterial strain(s) for use according to claim 13, wherein the pharmaceutically acceptable formulation is a medicament.

15. The bacterial strain(s) for use according to claim 13, wherein the food product is a medical food product.

16. The bacterial strain(s) for use according to claim 2, wherein said bacterial strain(s) is combined with one or more antibiotics.

17. The bacterial strain(s) for use according to claim 3, wherein said bacterial strain(s) is combined with one or more antibiotics.

18. The bacterial strain(s) for use according to claim 4, wherein said bacterial strain(s) is combined with one or more antibiotics.

19. The bacterial strain(s) for use according to claim 5, wherein said bacterial strain(s) is combined with one or more antibiotics.

20. The bacterial strain(s) for use according to claim 2, wherein the strain(s) is Lactobacillus acidophilus NCFM.


SEQUENCE LISTING (20 sequences)

SEQ ID NO: 1 - DNA, 22 bp, synthetic construct
  source 1..22: mol_type = other DNA
cccagggacctctctctaatca

SEQ ID NO: 2 - DNA, 20 bp, synthetic construct
  source 1..20: mol_type = other DNA
gctacaggcttgtcactcgg

SEQ ID NO: 3 - DNA, 23 bp, synthetic construct
  source 1..23: mol_type = other DNA
actgagagtgattgagagtggac

SEQ ID NO: 4 - DNA, 21 bp, synthetic construct
  source 1..21: mol_type = other DNA
aaccctctgcacccagttttc

SEQ ID NO: 5 - DNA, 23 bp, synthetic construct
  source 1..23: mol_type = other DNA
ggtatcgtggaaggactcatgac

SEQ ID NO: 6 - DNA, 23 bp, synthetic construct
  source 1..23: mol_type = other DNA
atgccagtgagcttcccgttcag

SEQ ID NO: 7 - DNA, 19 bp, synthetic construct
  source 1..19: mol_type = other DNA
gctggtgcgattggcttta

SEQ ID NO: 8 - DNA, 21 bp, synthetic construct
  source 1..21: mol_type = other DNA
ggatagcgacttgcacatcgt

SEQ ID NO: 9 - DNA, 25 bp, synthetic construct
  source 1..25: mol_type = other DNA
gcccacttctacagaaccgacatac

SEQ ID NO: 10 - DNA, 25 bp, synthetic construct
  source 1..25: mol_type = other DNA
aggcgataacgacaacttcaggatc

SEQ ID NO: 11 - DNA, 20 bp, synthetic construct
  source 1..20: mol_type = other DNA
ccagggagatcctttgatga

SEQ ID NO: 12 - DNA, 20 bp, synthetic construct
  source 1..20: mol_type = other DNA
aactggccacagttttcagg

SEQ ID NO: 13 - DNA, 20 bp, synthetic construct
  source 1..20: mol_type = other DNA
ggtctcaacccccagctagt

SEQ ID NO: 14 - DNA, 23 bp, synthetic construct
  source 1..23: mol_type = other DNA
gccgatgatctctctcaagtgat

SEQ ID NO: 15 - DNA, 24 bp, synthetic construct
  source 1..24: mol_type = other DNA
caggccatcagcaacaacataagc

SEQ ID NO: 16 - DNA, 21 bp, synthetic construct
  source 1..21: mol_type = other DNA
agctggtggaccactcggatg

SEQ ID NO: 17 - DNA, 20 bp, synthetic construct
  source 1..20: mol_type = other DNA
cggcaatgaagcttctgtat

SEQ ID NO: 18 - DNA, 20 bp, synthetic construct
  source 1..20: mol_type = other DNA
ccttgaaactctttgcctca

SEQ ID NO: 19 - DNA, 18 bp, synthetic construct
  source 1..18: mol_type = other DNA
gctgagtatgtcgtggag

SEQ ID NO: 20 - DNA, 19 bp, synthetic construct
  source 1..19: mol_type = other DNA
tcttctgagtggcagtgat